(19) United States
(12) Patent Application Publication
(10) Pub. No.: US 2026/0053688 A1
(43) Pub. Date: Feb. 26, 2026

(54) DYNAMIC HALO GRAVITY TRACTION SYSTEM AND METHOD

(71) Applicant: The Research Institute at Nationwide Children's Hospital, Columbus, OH (US)

(72) Inventors: Reid Chambers, New Albany, OH (US); Crystal Seilhamer, Ashville, OH (US); Holly Deem, Thornville, OH (US)

(21) Appl. No.: 19/105,591

(22) PCT Filed: Aug. 23, 2023
(86) PCT No.: PCT/US2023/030952

§ 371 Date: Feb. 21, 2025

Related U.S. Application Data
(60) Provisional application No. 63/400,204, filed on Aug. 23, 2022.

(51) Int. Cl. A61H 1/02; A61B 17/64; A61B 90/00; A61H 3/00

(52) U.S. Cl. CPC A61H 1/0229; A61B 17/6433; A61B 90/06; A61H 3/008; A61B 2090/064; A61H 2201/0107

(57) ABSTRACT

A modular dynamic halo gravity traction assembly and method of use is provided. The assembly includes a modular halo assembly for reducing spinal deformity of a user having a halo portion coupled to a force adjustment assembly connected to one of a first and second support members configured to support a portion of the body of the user in an upright position during use. The first and second support members are coupled by a connecting support member and the halo portion is in fluid connection with the connecting support member. A biasing system including a biasing member disposed within a biasing member housing is coupled to one of the support members and a force adjustment assembly having a fixture is movably coupled to said biasing housing, with a force adjustment rod having a proximal and a distal end, the ends coupled to the biasing housing.

### CROSS REFERENCES TO RELATED APPLICATIONS

The following application claims priority under 35 U.S.C. § 119 (e) to U.S. Provisional Patent Application Ser. No. 63/400,204 filed Aug. 23, 2022 entitled DYNAMIC HALO GRAVITY TRACTION AND METHOD OF USE. The above-identified application is incorporated herein by reference in its entirety for all purposes.

### TECHNICAL FIELD

The present disclosure generally relates to a dynamic halo gravity traction system and method of use, and more particularly to a modular halo assembly that includes a force adjusting assembly for altering tension in the system.

### BACKGROUND

Traditional halo gravity traction is used treat severe spinal deformities by gradually lengthening and straightening the spinal column of a user in preparation for spinal fusion surgery. The gradual correction allows the spine to be manipulated in a manner that is safe for the spinal cord. The process typically begins with the surgical insertion of pins to anterior and posterior locations on a patient's head. A carbon fiber halo ring is affixed through these pins, and the halo is then attached to a traction device that includes a pulley system.

The application of a halo for stabilization of the spinal column in factures as well as its use in the treatment of spinal deformities began in the early 1950s. Over the last half century, the use of halo traction to treat severe scoliosis has been demonstrated to be safe and effective. Recent publications have attempted to describe methods to incorporate the use of halo gravity traction into a practice. Particularly, the publication Halo Gravity Traction for Severe Pediatric Spinal Deformity: A Clinical Concepts Review is incorporated herein by reference in its entirety for all purposes.

Traditional halo gravity traction devices require a variety of apparatuses and skills to apply safely and effectively. There is no current system or vendor that provides a complete traction device which includes the traction rig, weights, and education/direction for safe and proper use. With a piecemeal approach, there are inherent safety risks to the patient and caregiver. Difficulty is substantially increased when the traction device is paired with wheelchair and/or walker modifications needed for the large majority of patients treated with this technique and technology. Traditional traction device management is difficult to manage outside of a hospital setting or without trained professionals. The counter-weights traditionally used to balance the traction device pose a safety risk for untrained patient caretakers. Furthermore, traditional methods of halo gravity traction may require irreversible modifications to patient walkers or wheelchairs, creating even more burden and cost before the patient undergoes the spinal fusion surgery that typically follows use of the traction device.

### SUMMARY

One aspect of the present disclosure includes a modular halo assembly for reducing spinal deformity of a user having a halo portion comprising ring, first and second support members configured to support a portion of the body of the user in an upright position during use, the first and second support members coupled and spaced by a connecting support member; the halo portion in fluid connection with the connecting support member, a biasing assembly comprising a biasing member disposed within a biasing member housing that is coupled to one of the support members, and a force adjustment assembly comprising a fixture movably coupled to said biasing member housing, a force adjustment rod having a proximal and a distal end, the ends coupled to the biasing housing.

Another aspect of the present disclosure includes a modular halo assembly for reducing spinal deformity of a user having a halo portion having a ring, first and second support members that are configured to support a portion of the body of the user in an upright position during use. The first and second support members coupled by a connecting support member. The halo portion is movably coupled with the connecting support member by a support line. A biasing arrangement having an inline biasing member disposed within a biasing member housing that is coupled to one of the support members by the support line, and a force adjustment assembly having a fixture movably coupled to the biasing housing, a force adjustment rod having a proximal and a distal end, the ends coupled to the biasing housing.

While yet another aspect of the present disclosure includes a modular and mobile halo traction system comprising a halo ring coupled to a pulley system movably connected to a support assembly. The halo ring provides dynamic traction coupling of a patient's cranium during use of the system. First and second support members are configured to support a portion of the body of the patient in an upright position during use is provided as well, the first and second support members spaced and coupled by a connecting support member wherein the halo portion is movably connected with the connecting support member via the pulley system. A biasing system has a biasing member disposed within a biasing member housing that is coupled to one of the support members and the pulley system. A force adjustment assembly is provided having a fixture plate movably selectively coupled to the biasing housing, the fixture plate being fixedly attached to the biasing member, and a tension selection pin for coupling the fixture plate and biasing member to a desired tension with the biasing member housing.

While an additional aspect of the present disclosure includes a method of providing modular and mobile halo traction system for reducing spinal deformity of a user, the method comprising the steps of: suspending a halo portion having a ring for supporting a portion of a patient's body during use with a first and second support members; coupling and spacing the first and second support members with a connecting support member; the halo portion movably coupled with the connecting support member by a support line; providing a biasing arrangement having an inline biasing member disposed within a biasing member housing that is coupled to one of the support members by the support line; and adjusting the tension in the biasing arrangement with a support plate that is coupled to the biasing member within the biasing member housing.

### BRIEF DESCRIPTION OF THE DRAWINGS

The foregoing and other features and advantages of the present disclosure will become apparent to one skilled in the art to which the present disclosure relates upon consideration of the following description of the disclosure with reference to the accompanying drawings, wherein like reference numerals refer to like parts unless described otherwise throughout the drawings and in which:

FIG. 1 is a modular halo assembly in accordance with one example embodiment of the present disclosure;

FIG. 2 is an in vivo view of a modular halo assembly coupled to a wheelchair in accordance with one example embodiment of the present disclosure;

FIG. 3 is an in vivo view of a modular halo assembly coupled to a walker in accordance with one example embodiment of the present disclosure;

FIG. 4 illustrates a flow diagram for a method of use of a modular halo assembly on a user in accordance with one example embodiment of the present disclosure;

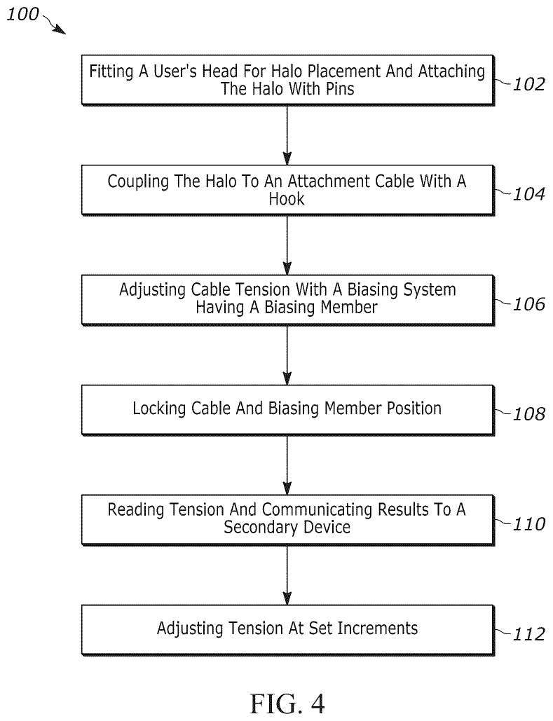

FIG. 5 is a schematic diagram of a tension meter reading communication system in accordance with one example embodiment of the present disclosure.

FIG. 6 is a top plan view of a modular halo assembly in accordance with one example embodiment of the present disclosure;

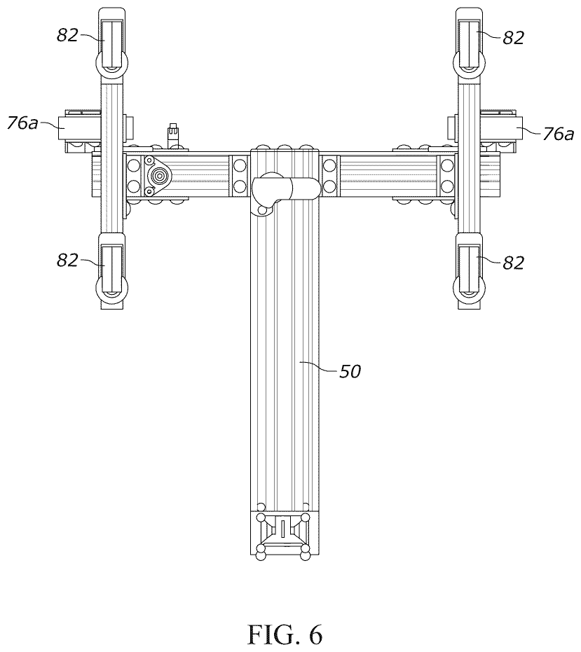

FIG. 7 is a bottom plan view of a modular halo assembly in accordance with one example embodiment of the present disclosure;

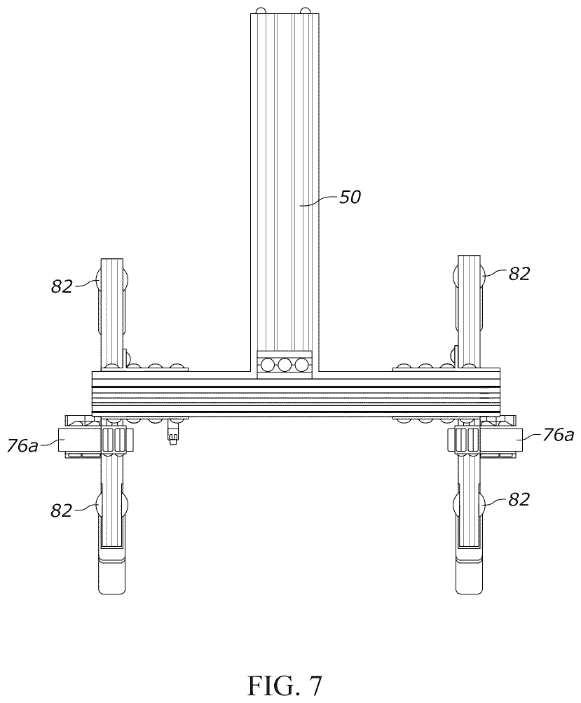

FIG. 8 is a front view of a force adjustment assembly in a first adjusted position in accordance with one example embodiment of the present disclosure;

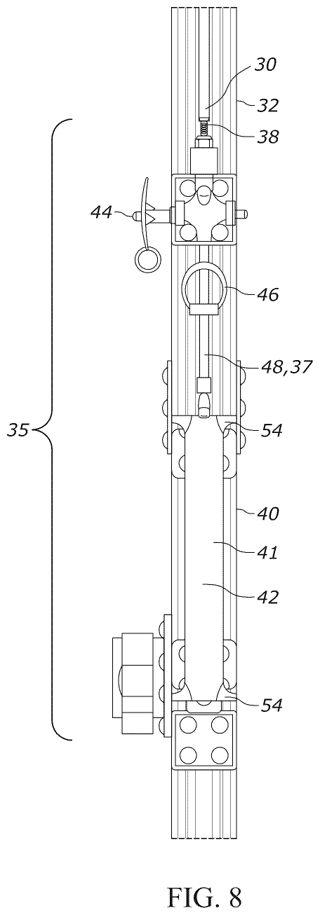

FIG. 9 is a front view of a force adjustment assembly in a second adjusted position in accordance with one example embodiment of the present disclosure;

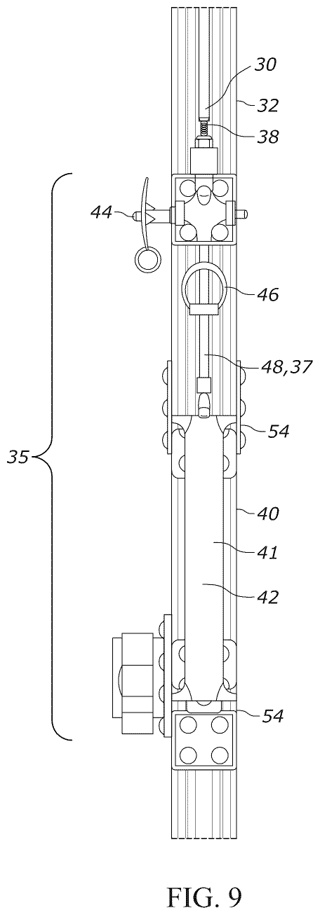

FIG. 10 is a front magnified view of a force adjustment assembly in a third adjusted position in accordance with one example embodiment of the present disclosure;

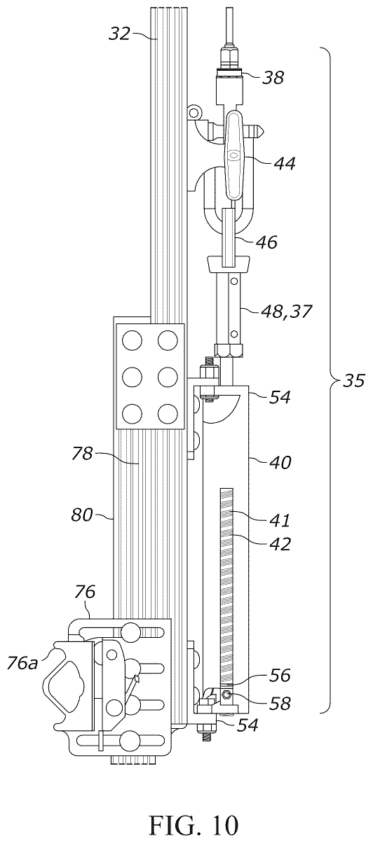

FIG. 11 is a front view of a force adjustment assembly in the third adjusted position in accordance with one example embodiment of the present disclosure;

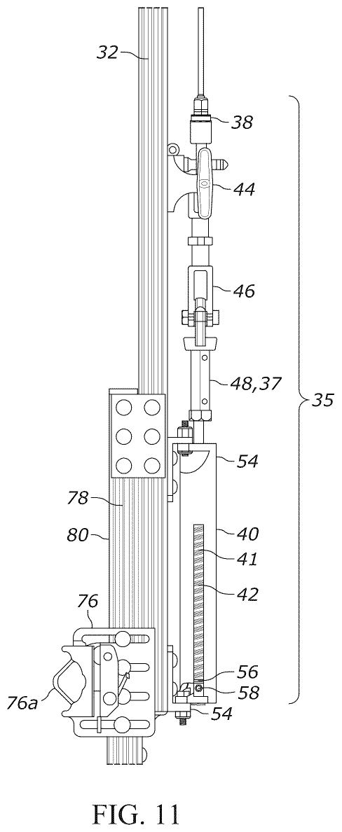

FIG. 12 is a front view of a connection bracket in accordance with one example embodiment of the present disclosure;

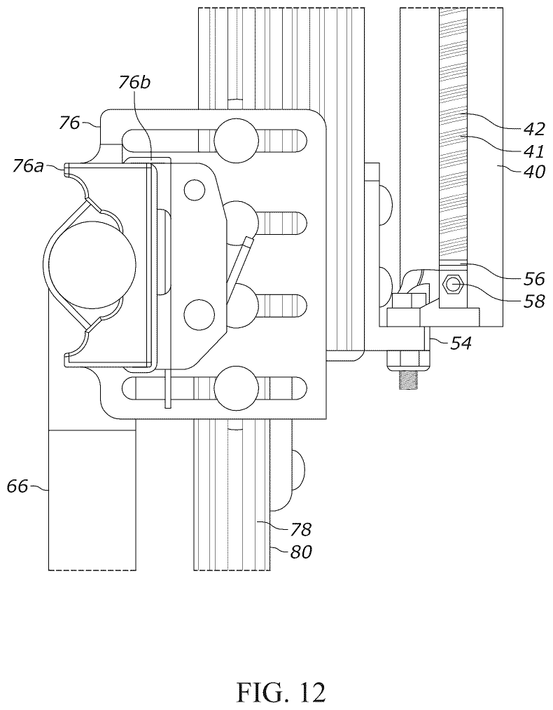

FIG. 13 is a front perspective view of a connection bracket in accordance with one example embodiment of the present disclosure;

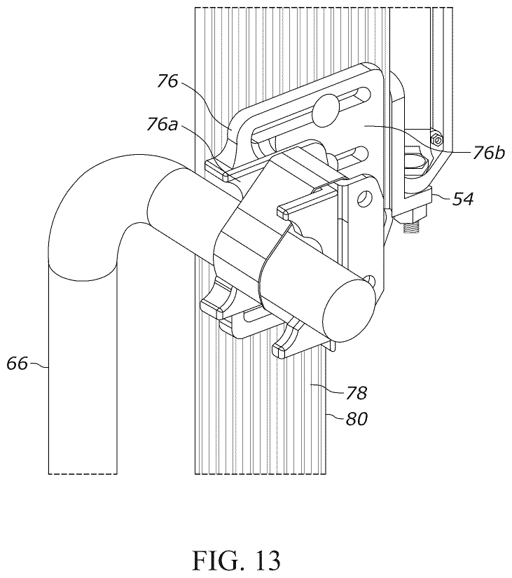

FIG. 14 is a front view of first and second support rods in accordance with one example embodiment of the present disclosure; and

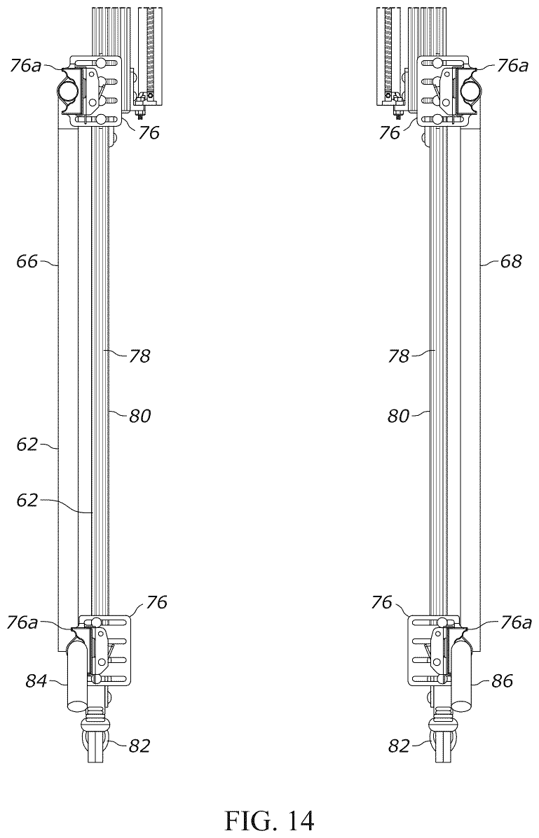

FIG. 15 is a front view of a balance extension in accordance with one example embodiment of the present disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

The apparatus and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION

Referring now to the figures generally wherein like numbered features shown therein refer to like elements throughout unless otherwise noted. The present disclosure relates to a dynamic halo gravity traction system and method of use, and more particularly to a modular halo assembly that includes a force adjusting assembly for altering tension in the system. The traction system and method of use is a mobile system that includes a cantilever support that allows a patient during use to substantially center a patient center of gravity while using a mobility device such as a walker or wheel chair during treatment by the system.

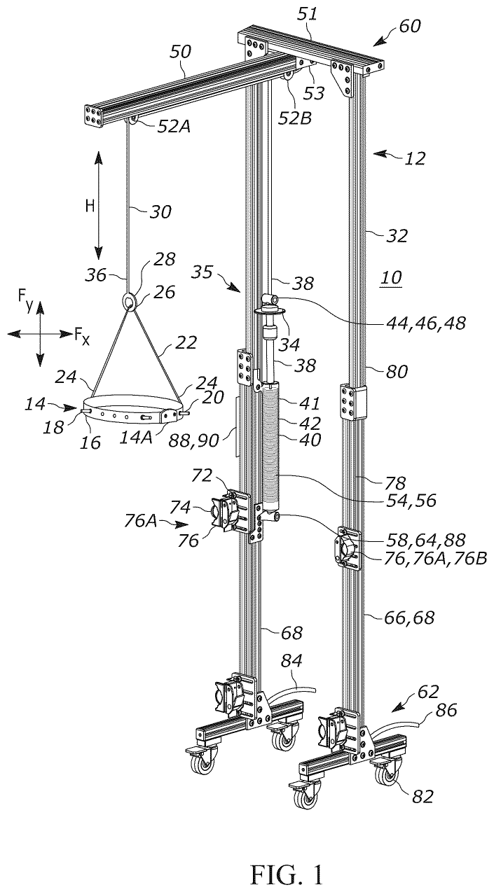

FIG. 1 illustrates a dynamic halo gravity traction system 10 of a modular halo assembly 12 in accordance with one example embodiment of the present disclosure. The individual parts that make up the modular halo assembly 12 may be generated from a variety of methods, such as 3D printing, extrusion, and/or an assembly line for commercial manufacturing. Unless specified otherwise, the modular halo assembly 12 is made from metal such as stainless steel or aluminum, plastics, or any combination thereof as would be appreciated by one of ordinary skill in the art. However, other materials of similar strengths or methods of manufacturing could be used.

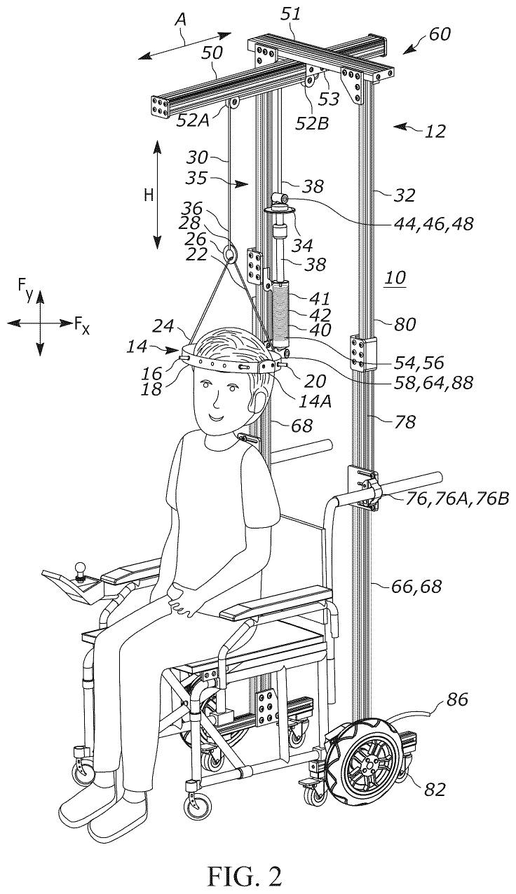

FIG. 2 includes a halo portion 14 of a modular halo assembly 12 in vivos with a user. The halo portion 14 is constructed from carbon fiber in this example embodiment, and a metal halo and/plastic and/or any combination thereof is also contemplated. In one example embodiment, a semi-annular, ring, or annular shape may be used for the halo portion 14. The halo 14 is coupled to the skull of a patient with pins 16 at anterior 18 and posterior 20 pin sites that form a holding contact connection with a patient's skull during use. The halo portion 14 further comprises a medial band 22 which is attached to the halo in two opposing attachment positions 24 located on either side of the sagittal plane SP of the patient. The medial band 22 may be constructed from carbon fiber, from a metal such as stainless steel, aluminum, or rubber, silicone, plastic, any combination thereof, and the like. The shape of the medial band 22 may vary, with possibilities including an arch shape extending across the skull of the user along a coronal plane CP, or a more triangular shape with a peak at a parallel location with the sagittal plane SP designated on the user or a combination thereof. The medial band 22 includes a hook or coupler 26, which is connectable to a clip 28 of a pulley system attachment line 30 of the modular halo assembly 12.

The pulley system attachment line 30 is supported by the assembly frame 32. The attachment line 30 comprises first and second ends 36, 38, respectively, the first end 36 is coupled to clip 28 that attaches to the halo 14. The second end 38 is coupled to connection portion 34 that pairs the attachment line 30 to a force adjustment assembly 35 comprising an inline biasing member housing 40, which at least partially houses a biasing member 41. In one example embodiment, the biasing member 41 is a spring, dash pod, dampening member, a plurality of each in series or parallel, or any combination thereof, forming a force response arrangement of the force adjustment assembly 35.

The pulley system attachment line 30 is connected to the modular halo assembly 10, slowly altering the position of the patient's spinal column when under load of the force adjustment assembly 35. The attachment line 30 may comprise a metallic braided cable, a wire, a fibrous rope, polymeric rope, or any other similar material with similar tensile strength of a one-quarter inch diameter metallic cable.

Also forming a part of the force adjustment assembly 35 is a handle 44, such as a twist handle that allows for the locking of the attachment line 26 into the connection portion 34 and allows for stabilization of the pulley system attachment line. The handle 44 further comprises an attachment ring 46 coupled to a turn buckle 48. The turn buckle 48 allows for fine adjustments to be made in the positioning and/or tension of the pulley system attachment line 30 and force adjustment assembly 35.

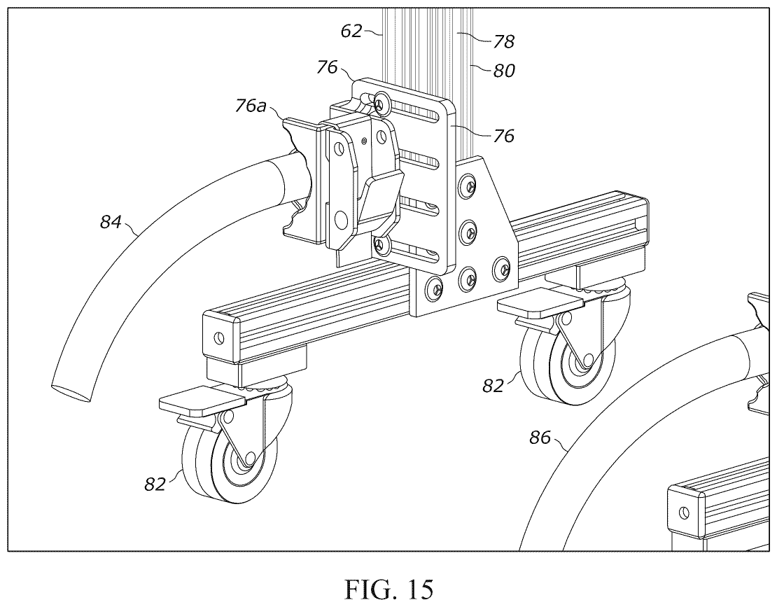

The assembly frame 32 forms a substantially angular arched shape that frames the body of a user, although an alternative shape may be feasible. The assembly frame 32 is constructed from steel, aluminum, or a similarly durable metal, with a nonmetal frame also being plausible, as appreciated by one of ordinary skill of the art. The assembly frame 32 may be constructed through various methods of extrusion to create individual metal members that may be bolted together to form assembly frame 32 with attachment brackets 32A (see FIG. 15). Alternatively, the metal members that comprise assembly frame 32 may be connected with corner pieces (not shown), or steel, aluminum, or other durable metal may be molded to allow for the frame to be made from one continuous piece of metal folded into a frame shape.

It is contemplated that the assembly frame 32 could be built for differing heights and/or widths for varying groups of users, such as pediatric patients and adults. In the preferred embodiment, the assembly frame 32 features a cantilevered portion 50 along which attachment line 30 runs. The cantilevered portion 50 has a proximal end which is coupled to the assembly frame 32 and a distal end extending outward, with the entirety of the cantilevered portion 50 providing a central point on the modular halo assembly 12 from which the attachment line 30 may reach the user. A first pulley wheel 52A is located at the distal end of the cantilevered portion 50 which supports the attachment line 30. The first pulley wheel 52A includes a groove 53A over which the attachment line 30 travels. The first pulley wheel 52A allows the attachment line 30 to provide a lifting force to the modular halo assembly 12.

The assembly frame 32 runs parallel with attachment line 30 subsequent to the line 30 passing over second pulley wheel 52B, located within one of the 90-degree angles created by the corners of assembly frame 32. The assembly frame 32 is coupled to the force adjustment assembly 35 and more specifically, a biasing member 41. In this example embodiment, the biasing member 41 comprises an inline spring 42 that may comprise a metal coil, although a nonmetal alternative may be possible. The inline spring 42 may comprise varying degrees of thickness and/or spring constants, depending on the severity of the user's spinal deformity and the elasticity needed from the spring 42, having a spring constant typical of a spinal traction system. In one example embodiment the spring constant ranges between one (1) pound force per linear foot to thirty (30) pounds force per linear foot. A spring 42 constructed with the aforementioned spring constant has a length that provides enough extension to support the body of a user and enough tension to alter the curvature of the user's spine. Although the current embodiment uses one inline spring 42, alternative embodiments are feasible with an additional inline spring of the same or varying spring constants in series or parallel. In yet another example embodiment, a dash pod or dampener is contemplated to be used in series or parallel with or without said spring or springs 42.

In the illustrated example embodiment, the inline spring 42 acts as a conduit for the transmission and storage of a restoring force F. Advantageously, the use of an inline springs 42 allows for an increase in patient and caregiver safety, as well as the capacity for at-home use of the halo gravity traction in some situations. The present disclosure could allow for a higher degree of portability in comparison to the prior art halo gravity traction systems. For example, a patient may be able to schedule medical visits for adjustment of the tension of the pulley system, and then return home with the device without danger or precarious storage or positioning of counterweights. Moreover, in the illustrated example embodiment, the tension can be adjusted to the patient through the system 10 by increasing or decreasing the position of force adjustment assembly 35 along the first or second support member 66 or 68. That is, the closer to the force adjustment assembly 35 is to the cantilever portion 50, the less the tension in the system 10 and force on the patient's spine and vice versa.

The prior art halo gravity traction assemblies included counterweights suspended on a posterior side of the halo gravity traction assembly as a main component in altering the curve of a spinal column of a user. These prior art assemblies combined the use of a halo with incremental additions of weight suspended from the anterior side of the assembly in order to lengthen the spinal column of the patient gradually over time, with the weight eventually being increased to 50% of the patient's body weight. The counterweights provided force that gradually pulled the patient's spinal column from an undulated line to a straight, vertical column. Difficulties were presented in balancing and changing the weights in terms of safety possibly for non-medically trained personnel. A patient using a traditional assembly would need to stay in the hospital for the entirety of the time the traction assembly is needed, usually around three to eight weeks, without the ability to leave the premises where the traction assembly was attached, due to the lack of portability of the device. Furthermore, many prior art traction devices required modification to wheelchairs and/or walkers used by the patient that could not be reversed upon completion of the halo gravity traction.

Stated another way, the inline spring of housing 40 of the present disclosure is attached with clamping fixtures 54 at either end of the spring housing and can be adjusted by movement of a spring base plate 56 located underneath and coupled to the bottom of the biasing member 41 or spring 42 inside of the spring housing. A pin 58 extending out of the spring housing 40 allows for adjustment of the position of the spring 42 and spring base plate 56 relative to the spring housing 40. That is, the spring housing 40 has several holes along its longitudinal axis for the inserting of the pin 58 thereby holding the spring base plate 56 and bottom of the spring 42 in a selectively adjustable fixed position. When secured by the pin 58, the spring 42 is permitted to expand when put under a load by the halo and weight or a portion of weight provided by the patient during use. It is desired that the force be adjusted based on the treatment and size that varies from patient to patient.

When the pin 58 is removed, the plate 56 secured to the bottom of the biasing member 41, or in this example spring 42 are free to move longitudinally along a central axis of the housing 40. As the plate 56 and biasing member 41 move upward within the housing, the less the range (as the biasing member expands and contracts) of the force or tension in the cable 30, force adjustment assembly 35, and the system 10 when the spring and base plate are resecured to the housing by the pin 58. When the desired tension range is found, for example with a force spring scale coupled to the halo 12 at the relative height of the halo for the patient's size and treatment, the pin 58 that holds the plate 56 into is inserted into the housing 40 so the bottom of the biasing member 41 or spring 42 is selectively adjustably fixed.

When the pin 58 is removed, the spring base plate 56 secured to the bottom of the biasing member 41, or in this example spring 42 are free to move longitudinally along a central axis of the housing 40. As the plate 56 and biasing member 41 transition downward within the housing, the greater the range (as the biasing member expands and contracts) of the force or tension in the cable 30, force adjustment assembly 35, and the system 10 when the spring and base plate are resecured to the housing 40. When the desired tension range is found, for example with a force spring scale coupled to the halo 12 at the relative height of the halo for the patient's size and treatment, the pin 58 that holds the plate 56 into position is inserted into the housing 40 so the bottom of the biasing member 41 or spring 42 is selectively adjustably fixed.

This first tension adjustment of the system 10 described above for increasing and decreasing the tension in the force adjustment assembly 35 is advantageous because the assembly 35 is completely self-contained as part of the system 10 and provides for mobility. Moreover, the force adjustment assembly 35 does not require any additional weights or remote parts. The force adjustment assembly 35 through the pin 58 removal and insertion allows for quick change in the system 10 when desired. Often the force in the system 10 needs to be changed based on the weight and size that varies among patients.

The inline spring 42 can hold a variety of positions, including a fully extended and fully compressed position. Positioning of the pin 58 nearest to a superior end 60 of the assembly frame 32 directs the spring 42 into a compressed position comprising restoring force F. Positioning of the pin 58 toward an inferior end 62 of the assembly frame 32 spreads restoring force F across a body 64 of the inline spring 42, increasing the amount of restoring force F contained in the inline spring 42 from the fully compressed position. Locking the inline spring 42 into the fully compressed position results in less tension of the attachment line 30 because the restoring force F is less than the amount of restoring force F available in the fully extended position.

Locking the inline spring 42 into the fully extended position results in a maximum amount of tension on the attachment line 30, and, thus, the user's spine, because there is more restoring force F generated through the stretching and locking of the inline spring 42 compared to what is available in the fully compressed position. Stated another way, the greater the compression of the inline spring 42, the less tension is placed on the spine of the user, due to less restoring force F provided by the inline spring 42.

The inline spring 42, with the assistance of first and second pulley wheels 52A, 52B, provide restoring force F in the opposite direction and equivalent to the displacement (stress a) caused by the weight of the user on the halo and particularly the anterior side of the modular halo assembly 12. The stress a applied to the attachment line 30, which carries the weight of the body of the user, is balanced by the restoring force F provided by the inline spring 42. A gradual shift from the compressed position, in which the inline spring 42 holds a minimum amount of tension, to the extended position, in which the inline spring 42 holds a maximum amount of tension, should be made over an entirety of the time period that the user is in the modular halo assembly 12, anywhere from 3-8 weeks. The attachment line 30 and its associated forces F resulting from the body of the user on the anterior side and the inline spring 42 on the posterior side generates orthogonal vector forces Fx, Fy.

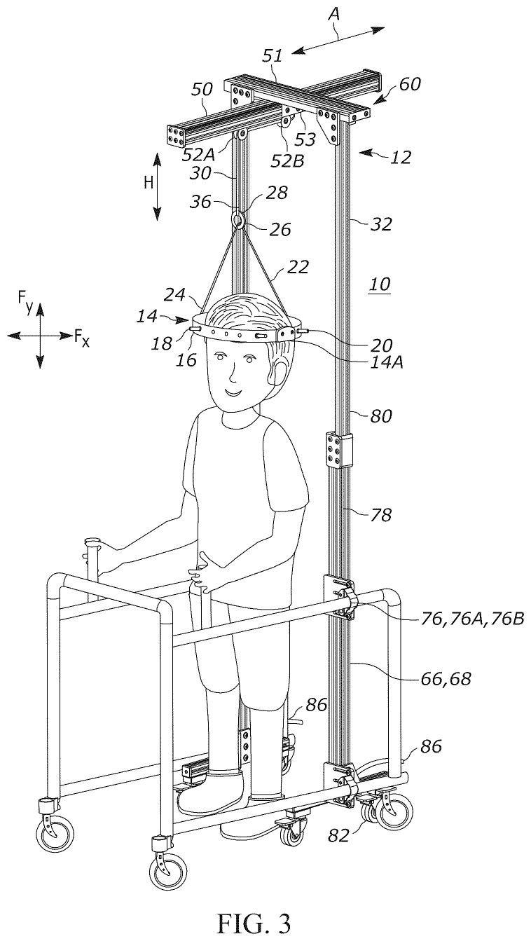

In another example embodiment, the track supports connection brackets 76, which may be anchored with a fastener or a plurality of fasteners. The connection brackets 76 include handles 76A that may grip the bars of a walker or the handles of a wheelchair, as shown in FIGS. 2 and 3. Since the connection brackets 76 are anchored onto embedded grooves 78 of a length of track 80 along first and second support members 66, 68, of the frame 32, the brackets 76 may be adjusted freely based on height. The handles 76A may comprise the aluminum, steel, or other metal or nonmetal from which the bracket 76 is made, and be molded as part of the bracket 76. Alternatively, the handles 76A may be external to the connection bracket and wrap around a groove 76B of the bracket 76. The handles 76A in this example embodiment may be comprised of Velcro strips, rubber, or any other flexible and secure attachment method or any other material capable of gripping and holding the walker or wheelchair into place, as would be appreciated by one of ordinary skill in the art.

In another example embodiment, the handles 76A partially house first and second support members 66, 68, respectively that are coupled to the inferior portion 62 of the frame 32 in a second set of connection brackets 76. The first and second support members 66, 68 extend from a medial portion 70 of the assembly frame arch 32 to the inferior portion of the frame 62. The support members 66, 68 comprise arcuate lines running parallel with assembly frame 32. The frame 32 is further secured by said support members 66, 68, especially in cases where the user does not use a wheelchair or walker in conjunction with the modular halo assembly 12. The first and second support members 66, 68 are also constructed from steel, aluminum, or an equally durable metal or nonmetal material, as appreciated by one of ordinary skill in the art.

In the illustrated example embodiment, the first and second support members 66 and 68 are coupled to and spaced by a connecting member 51. The connecting member 51 includes a slide 53 that is coupled to tracks of the cantilever portion 50 that allows for the lateral movement of the cantilever portion 50 (indicated by arrows A in FIGS. 2-3) and halo portion 14 relative to the first and second support members 66, 68. This allows adjustment for the patient's size and location when using a walker, wheelchair, standing, or walking with the system 10. It is contemplated that the width of the system 10 can be increased by increasing the length of the connecting portion 50. The slide 53 can lock the cantilever portion 50 into a selected position or move with the patient as prescribed by the medical professional.

At the inferior portion 62 of the modular halo assembly 12, support members 66, 68 support swivel caster wheels 82, which should be enabled to maintain a high load capacity in order to support the body of a patient. A variety of other types of wheels would also be compatible with the modular halo assembly 12, as appreciated by one of ordinary skill in the art. The wheels 82 are contemplated to have at least 90-degree rotation capacity, with the preferred embodiment having 360-degree rotation wheels allowing for maximum ease of movement and flexibility. Wheels without rotation capability in any combination could also be used in some circumstances. A plurality of two or four wheels 82 would add additional support and rotational capabilities.

The frame 32 may also further comprise balance extensions 84, 86. The balance extensions 84, 86 are located at the inferior portion 62 of the frame 32 and run substantially perpendicular with the track 80. The balance extensions 84, 86 comprise an arcuate shape, and run parallel with the ground without coming into contact with it. These extensions 84, 86 provide balancing assistance in situations in which the patient's spine inadvertently rotates backwards in the course of movement.

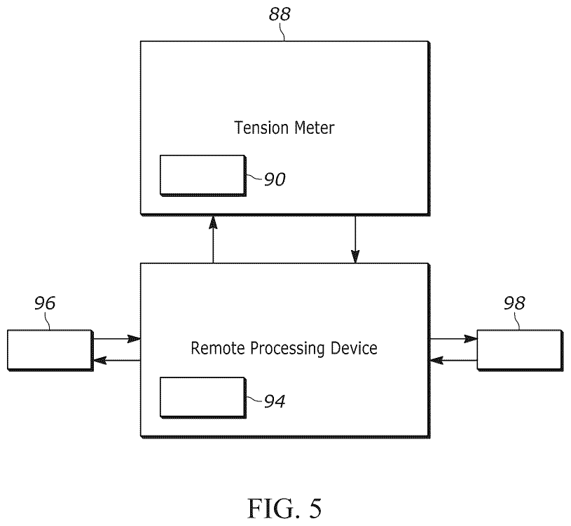

In another example embodiment, as illustrated in FIG. 5, the assembly 12 includes a tension meter 88 that may be enabled with a communication apparatus 90. The tension meter 88 may be analog or digitally operating. In this example embodiment, the tension meter 88 and communication apparatus 90 together read and communicate tension readings of the attachment line 30 with a remote processing device 92 through short wave radio waves (Bluetooth), Wi-Fi, Internet, wireless communication, or the like. The remote processing device 92 would generate outputs based on inputs from the communication apparatus 90. It would be appreciated by one having ordinary skill in the art that in some embodiments the remote processing device 92 would include a data storage device 94 in various forms of non-transitory, volatile, and non-volatile memories which would store buffered or permanent data as well as compiled programming codes used to execute functions of the remote processing device 92. In another example embodiment, the data storage device 94 can be external to and accessible by the remote processing device 92. In yet another example embodiment, the data storage device 94 includes an external hard drive, cloud storage, and/or other external recording devices 96.

In one example embodiment, the remote processing device 92 comprises one of a remote or local computer system. The computer system includes desktop, laptop, tablet hand-held personal computing device, IAN, WAN, WWW, and the like, running on any number of known operating systems and are accessible for communication with remote data storage, such as a cloud, host operating computer, via a world-wide-web or Internet.

After the tension meter 88 reads the tension of the pulley system attachment line 30, communication apparatus 90 transmits the reading to remote processing device 92 which communicates results with a secondary device 98 such as a smartphone or tablet. These communications could be scheduled in hourly, daily, or weekly increments, or the communication apparatus 90 may be set to send an alert if tension reaches or surpasses a set threshold. This feature allows the user's doctor to reaffirm patient safety, making sure that tension is within the ideal range for the particular stage of traction. The tension meter 88 enabled with a communication device 90 is one of many features that allows the present disclosure to be portable in some circumstances.

A typical use of the modular halo assembly 12 will now be described. The first step 102 of a method of use 100 of a traction device is fitting a user's head for a halo 14 and attaching said halo 14 with pins 16 in anterior and posterior positions 18, 20 of the user's skull. Next, in step 104, the halo 14 is coupled to an attachment cable 30 through a hook 26 and clip 28 or equivalent attachment system. Subsequently, in the third step 106, cable tension is adjusted with a biasing system having a biasing member 41, with the preferred embodiment comprising pulley wheels 52A, 52B and inline spring 42 system. In the fourth step 108, the cable 30 and biasing member 41 positions are locked into place. In an embodiment of the modular halo assembly 12 that includes a communication device 90, an additional step 110 occurs subsequent to the locking of the cable 30 and inline spring 42 position 108. A tension reading is performed by a tension meter 88 and subsequently communicated to a remote processing device 92 that may be in communication with a secondary device 98. This step may be performed at some frequency, such as daily, weekly, hourly, or on an as-needed basis, with “as-needed” meaning that the tension of the attachment line 30 has fallen outside of a set range of acceptable tension. Finally, in step 112, the tension is adjusted through a repetition of the last two steps throughout the duration of the user's use of the modular halo assembly 12 at set increments. The tension meter 88 could be any type of load cell having a transceiver to communicate the tension values to a remote device, such as a phone, Internet, computer, and the like.

In the foregoing specification, specific embodiments have been described. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The disclosure is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. In one non-limiting embodiment the terms are defined to be within for example 10%, in another possible embodiment within 5%, in another possible embodiment within 1%, and in another possible embodiment within 0.5%.

The term “coupled” as used herein is defined as connected or in contact either temporarily or permanently, although not necessarily directly and not necessarily mechanically. A device or structure that is “configured” in a certain way is configured in at least that way but may also be configured in ways that are not listed. The term “integral” as used herein unless defined otherwise means configured in such a way that separation would require destruction to the parts or the assembly of the parts.

It should be appreciated by those of ordinary skill in the art after having the opportunity of reviewing the drawings and/or specification of the present disclosure that it may include one or more embodiments, e.g., E₁, E₂, . . . Eₙ and that each embodiment E may have multiple parts A₁, B₁, C₁ . . . Zₙ that (without further description) could be combined with other embodiments Eₙ, embodiment parts e.g. A₁, C₁, or lack of parts originally associated with one or all embodiments Eₙ, or any combination of parts and/or embodiments thereof. It should further be appreciated that an embodiment Eₙ may include only one part e.g. A₁ or a lesser number of parts e.g. B₁, C₁ of any embodiment or combination of embodiments that was described or shown in the specification and/or drawings, respectively in ways not enumerated or illustrated.

To the extent that the materials for any of the foregoing embodiments or components thereof are not specified, it is to be appreciated that suitable materials would be known by one of ordinary skill in the art for the intended purposes after having the benefit of reviewing the subject disclosure and accompanying drawings.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

What is claimed is:

1. A modular halo assembly for reducing spinal deformity of a user comprising:
a halo portion comprising a ring;
first and second support members configured to support a portion of the body of a patient in an upright position during use, the first and second support members coupled by a connecting support member; the halo portion movably coupled with the connecting support member by a support line;
a biasing arrangement comprising an inline biasing member disposed within a biasing member housing that is coupled to one of the support members by said support line; and
a force adjustment assembly comprising a fixture movably coupled to said biasing member housing, a force adjustment rod having a proximal and a distal end, the ends coupled to the biasing member housing.

2. The modular halo assembly of claim 1 wherein the halo portion comprises an annular ring.

3. The modular halo assembly of claim 1, wherein each of the first and second support members are coupled to a respective wheel.

4. The modular halo assembly of claim 1, wherein the biasing member comprises an extension spring.

5. The modular halo assembly of claim 1, wherein said ring is continuous.

6. The modular halo assembly of claim 1 wherein said connecting support member further comprises a cantilever member for movable support of said halo portion so that during use, a patient can be substantially centrally located within the first and second support members to accommodate a patient mobility device.

7. The modular halo assembly of claim 1 further comprising a tension reading arrangement for measuring the tension forces or range of tension forces in the force adjustment assembly, the tension reading arrangement further comprising a transceiver for transmitting tension force data to a remote device.

8. The modular halo assembly of claim 7 wherein said tension reading arrangement further comprises a load cell.

9. A modular and mobile halo traction system comprising:
a halo ring coupled to a pulley system movably connected to a support assembly, the halo ring for dynamic traction coupling of a patient's cranium during use of the system;
first and second support members configured to support a portion of the body of the patient in an upright position during use, the first and second support members spaced and coupled by a connecting support member wherein the halo portion is movably connected with the connecting support member via said pulley system;
a biasing system comprising a biasing member disposed within a biasing member housing that is coupled to one of the support members and said pulley system; and
a force adjustment assembly comprising a fixture plate movably selectively coupled to said biasing housing, and said fixture plate being fixedly attached to said biasing member, and a tension selection pin for coupling said fixture plate and biasing member to a desired tension with said biasing member housing.

10. The modular and mobile halo traction system of claim 9, wherein each of the first and second support members are coupled to a respective wheel.

11. The modular and mobile halo traction system of claim 9, wherein the biasing member comprises an extension spring.

12. The modular and mobile halo traction system of claim 9, wherein said ring is continuous.

13. The modular and mobile halo traction system of claim 9 wherein said connecting support member further comprises a cantilever member for movable support of said halo ring so that during use, the patient can be substantially centrally located within the first and second support members to accommodate a patient mobility device.

14. The modular and mobile halo traction system of claim 9 further comprising a tension reading arrangement for measuring the tension forces or range of tension forces in the force adjustment assembly, the tension reading arrangement further comprising a transceiver for transmitting tension force data to a remote device.

15. The modular and mobile halo traction system of claim 14 wherein said tension reading arrangement further comprises a load cell.

16. A method of providing modular and mobile halo traction system for reducing spinal deformity of a user, the method comprising the steps of:
suspending a halo portion comprising a ring for supporting a portion of a patient's body during use with a first and second support members;
coupling and spacing said first and second support members with a connecting support member; the halo portion movably coupled with the connecting support member by a support line;
providing a biasing arrangement comprising an inline biasing member disposed within a biasing member housing that is coupled to one of the support members by said support line; and
adjusting the tension in the biasing arrangement with a support plate that is coupled to the biasing member within the biasing member housing.

17. The method of claim 16 further comprising the step of coupling the first and second support members with a respective wheel.

18. The method of claim 16 further comprising the step of using an extension spring for said biasing member.

19. The method of claim 16 further comprising the step of providing a cantilever member to the connecting support member for movable support of said halo portion so that during use, the patient can be substantially centrally located within the first and second support members to accommodate a patient mobility device.

20. The method of claim 16 further comprises the step of providing a tension reading arrangement for measuring the tension forces or range of tension forces in the force adjustment assembly, and transmitting tension force data to a remote device from the tension reading arrangement with a transceiver located on said system.

21. The modular and mobile halo traction system of claim 9, wherein the first and second support members are removably couplable to a patient mobility device.
